(19) United States
(12) Patent Application Publication
(10) Pub. No.: US 2018/0306766 A1
(43) Pub. Date: Oct. 25, 2018

(54) OPTICAL ELEMENT INCLUDING AT LEAST ONE LENS, BIOLOGICAL INFORMATION MEASURING APPARATUS INCLUDING THE OPTICAL ELEMENT, AND ILLUMINATION APPARATUS INCLUDING THE OPTICAL ELEMENT

(71) Applicant: Panasonic Intellectual Property Management Co., Ltd., Osaka (JP)

(72) Inventors: TERUHIRO SHIONO, Osaka (JP); TAKAMASA ANDO, Osaka (JP)

(21) Appl. No.: 15/945,793

(22) Filed: Apr. 5, 2018

(30) Foreign Application Priority Data
Apr. 24, 2017 (JP) 2017-085091

(51) Int. Cl. G01N 33/48; G02B 27/09; G01N 21/84

(52) U.S. Cl. CPC G01N 33/48; G01N 21/84; G02B 27/0955

(57) ABSTRACT

A biological information measuring apparatus includes a light source that emits emission light with which a portion to be examined is to be irradiated, a photodetector that detects light returning from the portion to be examined, and an optical element that includes at least one lens and that is disposed in an optical path between the light source and the portion to be examined. At least one value selected from the group consisting of a thickness and a refractive index of the at least one lens varies along a first direction extending from a center portion of the at least one lens toward an outer edge portion, and the at least one value is locally minimum at the center portion and locally maximum at a first portion that lies between the center portion and the outer edge portion.

### BACKGROUND

### 1. Technical Field

The present application relates to an optical element, a biological information measuring apparatus including the optical element, and an illumination apparatus including the optical element.

### 2. Description of the Related Art

A biological information measuring apparatus is widely used that irradiates an organism with light, detects reflected scattered light from the inside of the organism, and can thus contactlessly obtain useful information on the organism.

Irradiation light enters into the organism through the skin, is transmitted through an internal organ, such as a blood vessel, and then emerges as scattered light. Thus, the scattered light includes biological information on the heartbeat, the blood flow volume, the blood pressure, the saturation of peripheral oxygen, and so on. Detecting this scattered light with a biological information measuring apparatus makes it possible to obtain, for example, information on the pulse, the blood flow, the oxygen saturation, and so on. These pieces of information can be used in a medical examination or the like.

Japanese Unexamined Patent Application Publication No. 2003-337102 discloses an apparatus for measuring biological activities, and this apparatus noninvasively measures biological activities, such as brain activities, indicating the functions of an organism. This apparatus includes a light source unit that generates infrared light, a light detecting unit that detects infrared light coming from a human body, and an optical system that controls a position on the human body at which the human body is irradiated with the light. This apparatus irradiates substantially the entirety of a human forehead with near-infrared light and receives reflected scattered light with a photodetector such as a charge coupled device (CCD).

### SUMMARY

In one general aspect, the techniques disclosed here feature a biological information measuring apparatus. The biological information measuring apparatus includes a light source that emits emission light with which a portion to be examined is to be irradiated; a photodetector that detects light returning from the portion to be examined, the light being produced when the portion to be examined is irradiated with the emission light; and an optical element that includes at least one lens, the optical element being disposed in an optical path between the light source and the portion to be examined. At least one value selected from the group consisting of a thickness of the at least one lens and a refractive index of the at least one lens varies along a first direction extending from a center portion toward an outer edge portion, the center portion being a portion that includes a center of the at least one lens, and the at least one value is locally minimum at the center portion and locally maximum at a first portion that lies between the center portion and the outer edge portion.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a schematic diagram for describing a configuration of a biological information measuring apparatus and how biological information is measured according to a first embodiment of the present disclosure;

FIG. 2A is a plan view schematically illustrating a configuration of an optical element according to the first embodiment of the present disclosure;

FIG. 2B is a sectional view taken along the IIB-IIB line indicated in FIG. 2A;

FIG. 3 is a graph illustrating a relationship between the sag amount of an optical element and the radius from the optical axis center according to the first embodiment of the present disclosure;

FIG. 4 is a diagram illustrating an intensity distribution (solid line) of emission light from a biological information measuring apparatus toward a portion to be examined along a plane perpendicular to the optical axis according to the first embodiment of the present disclosure and an intensity distribution (dotted line) of emission light along a plane perpendicular to the optical axis obtained when an optical element is not provided;

FIG. 5 is a graph illustrating a relationship between the sag amount of an optical element and the radius from the optical axis center according to a modification of the first embodiment of the present disclosure;

FIG. 6 is a diagram illustrating an intensity distribution (solid line) of emission light from a biological information measuring apparatus toward a portion to be examined according to the modification of the first embodiment of the present disclosure and an intensity distribution (dotted line) of emission light obtained when an optical element is not provided;

FIG. 7A is a plan view schematically illustrating a configuration of an optical element according to another modification of the first embodiment of the present disclosure;

FIG. 7B is a sectional view taken along the VIIB-VIIB line indicated in FIG. 7A;

FIG. 8A is a diagram for describing a configuration of a biological information measuring apparatus and how biological information is measured according to a second embodiment of the present disclosure;

FIG. 8B is another diagram for describing a configuration of a biological information measuring apparatus and how biological information is measured according to the second embodiment of the present disclosure;

FIG. 9A is a plan view schematically illustrating a configuration of an optical element according to the second embodiment of the present disclosure;

FIG. 9B is a sectional view taken along the IXB-IXB line indicated in FIG. 9A;

FIG. 9C is a sectional view taken along the IXC-IXC line indicated in FIG. 9A;

FIG. 10 is a graph illustrating distributions of the sag amounts of a lens in the X-direction and in the Y-direction according to the second embodiment of the present disclosure;

FIG. 11 is a diagram illustrating an intensity distribution (dashed-dotted line) in the X-direction and an intensity distribution (solid line) in the Y-direction of emission light from a biological information measuring apparatus toward a portion to be examined along a plane perpendicular to the optical axis according to the second embodiment of the present disclosure and an intensity distribution (dotted line) of emission light along a plane perpendicular to the optical axis obtained when an optical element is not provided;

FIG. 12 is a plan view schematically illustrating another configuration of an optical element according to the second embodiment of the present disclosure;

FIG. 13A is a plan view schematically illustrating a configuration of an optical element according to a modification of the second embodiment of the present disclosure;

FIG. 13B is a sectional view taken along the XIIIB-XIIIB line indicated in FIG. 13A;

FIG. 14A is a diagram for describing a configuration of a biological information measuring apparatus and how biological information is measured according to a third embodiment of the present disclosure;

FIG. 14B is another diagram for describing a configuration of a biological information measuring apparatus and how biological information is measured according to the third embodiment of the present disclosure;

FIG. 14C is a diagram for describing a portion to be examined according to the third embodiment of the present disclosure;

FIG. 15A is a plan view schematically illustrating a configuration of an optical element according to a modification of the third embodiment of the present disclosure;

FIG. 15B is a sectional view taken along the XVB-XVB line indicated in FIG. 15A;

FIG. 16A is a schematic diagram illustrating a situation in which a biological information measuring apparatus according to a comparative example irradiates a portion to be examined having a planar surface with light from a light source; and

FIG. 16B is a schematic diagram illustrating a situation in which the biological information measuring apparatus according to the comparative example irradiates a portion to be examined having a curved surface, such as a forehead, an arm, or a leg, with light from the light source.

### DETAILED DESCRIPTION

Prior to describing embodiments of the present disclosure, underlying knowledge for forming a basis of the present disclosure will be described.

The present inventors have found the following problem. Specifically, when biological information on a portion to be examined having a surface that is not planar, such as a forehead, an arm, or a leg, is to be acquired, the intensity of irradiation light becomes lower in a peripheral region of the portion to be examined than in a center region, which thus leads to a lower signal-to-noise ratio (S/N). Hereinafter, this problem will be described.

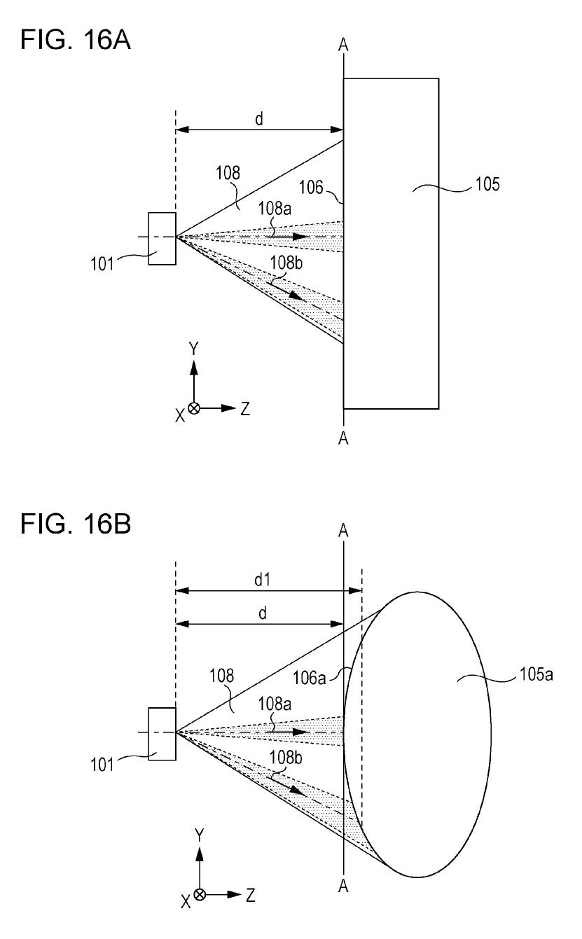

FIG. 16A is a schematic diagram illustrating a situation in which a biological information measuring apparatus according to a comparative example irradiates a portion to be examined having a planar surface with light from a light source 101. FIG. 16B is a schematic diagram illustrating a situation in which the biological information measuring apparatus according to the comparative example irradiates a portion to be examined having a curved surface, such as a forehead, an arm, or a leg, with light from the light source 101.

In the following descriptions, the XYZ-coordinates indicated in FIG. 16A and FIG. 16B will be used. The X-, Y-, and Z-directions are orthogonal to one another.

The biological information measuring apparatus illustrated in FIGS. 16A and 16B is disposed in proximity to a portion 106 to be examined of a biological subject 105 or in proximity to a portion 106a to be examined of a biological subject 105a. The distance d from the light source 101 to the center of the portion 106 to be examined or the portion 106a to be examined is short, and substantially the entirety of the light emitted by the light source 101 reaches the portion 106 to be examined or the portion 106a to be examined. The biological information measuring apparatus includes constituent elements such as a photodetector and a control circuit, but these are omitted from the drawings.

First, with reference to FIG. 16A, a case in which the planar portion 106 to be examined is irradiated with divergent light 108 emitted from the light source 101 will be described. Herein, a plane that is perpendicular to the center axis of the light emitted from the light source 101 and in which the surface of the portion 106 to be examined is located is referred to as an A-A plane. The light 108 emitted from a typical light source 101, such as a laser or a light emitting diode (LED), is a Gaussian beam having a Gaussian distribution. In a Gaussian beam, light 108a in the center portion has a high optical intensity, and light 108b in a peripheral portion has a low optical intensity. Therefore, a light spot formed in the A-A plane has a higher optical intensity at the center portion, and the optical intensity decreases as the distance from the center portion increases.

Unlike the case illustrated in FIG. 16A, when the distance d from the light source 101 to the portion 106 to be examined is sufficiently large, the portion 106 to be examined is irradiated with the light 108a in the center portion of the Gaussian distribution. Therefore, the optical intensity distribution on the portion 106 to be examined becomes substantially uniform. As a result, the S/N of a detected biological signal is substantially constant within the region of the portion 106 to be examined. In this case, however, of the light 108 emitted from the light source 101, the light 108b in the peripheral portion is incident on a region outside the portion 106 to be examined, and thus the light 108b is not used to acquire biological information. In this manner, a large distance d leads to a problem in that the utilization efficiency of the light decreases.

Meanwhile, when the distance d is small as illustrated in FIG. 16A, the light 108b in the peripheral portion of the Gaussian distribution is also incident on the portion 106 to be examined. Thus, the utilization efficiency of the light improves. However, the optical intensity of the light 108b in the peripheral portion is lower than the optical intensity of the light 108a in the center portion. As the intensity of the light 108 with which the portion 106 to be examined is irradiated is lower, the S/N of a detected biological signal tends to decrease. Therefore, the S/N of a biological signal from a peripheral region is lower than that of a biological signal from the center portion.

Next, an example in which a diffuser is disposed in an optical path between the light source 101 and the portion 106 to be examined at a position in proximity to the light source 101 to transform the light 108 into light with a Lambertian distribution will be described. The light 108 of the Lambertian distribution has a broad radiation angle, and the full width at half maximum of this radiation angle is 120 degrees. When the distance d is small, the light 108b in the peripheral portion is obliquely incident on the portion 106 to be examined. As the optical path length of the light 108b in the peripheral portion is increased, the intensity of the light 108b in the peripheral portion is lower than the intensity of the light 108a in the center portion in the A-A plane. As a result, although the S/N of a biological signal from the peripheral region improves due to the diffuser, the S/N is still lower than that of a biological signal from the center portion. Since the light 108b in the peripheral portion is obliquely incident on the portion 106 to be examined, the smaller the distance d is, the greater is the extent by which the S/N decreases.

When the distance d is relatively large-for example, when d=300 mm, the longitudinal and lateral sizes of the portion 106 to be examined are each 100 mm, and the optical intensity of the light 108a in the center portion is 1, the optical intensity of the light 108b in the outermost peripheral portion in the A-A plane is 0.97. In this manner, when the distance d is large, the optical intensity of the light 108b in the peripheral portion decreases to a lesser extent. In such a case, the use of a diffuser can solve the problem that the optical intensity decreases in the peripheral portion.

However, when the distanced is small-for example, when d=100 mm, the optical intensity of the light 108b in the outermost peripheral portion decreases to 0.74. Furthermore, when d=50 mm, this optical intensity decreases to 0.4. In this manner, it has been found that, as the distance d is smaller, the optical intensity in the peripheral portion decreases further.

Next, a case in which the curved portion 106a to be examined is irradiated with the divergent light 108 emitted from the light source 101, as illustrated in FIG. 16B, will be described. The optical intensity distribution in the A-A plane that passes through the center of the portion 106a to be examined and that is perpendicular to the center axis of the emission light is the same as in the case illustrated in FIG. 16A described above. However, since the portion 106a to be examined is curved, the optical path length of the light 108b incident on the peripheral portion of the portion 106a to be examined is further extended, and the divergence of the light 108 on the portion 106a to be examined increases accordingly. Therefore, the intensity of the light 108b in the peripheral portion of the curved portion 106a to be examined is further reduced than the intensity of the light 108a in the center portion. This results in the problem that the S/N of a signal indicating biological information of the peripheral region is also reduced to a greater extent.

Furthermore, the degree of the decrease in the S/N of a biological signal differs at different portions to be examined. For example, the shape of an arm or a leg is close to a column or a cylindroid, and the curvature of a forehead differs for the horizontal direction (X-direction) and for the vertical direction (Y-direction). Therefore, when such portions to be examined are to be measured, the degree of the decrease in the S/N of a biological signal from the peripheral region differs in the two orthogonal directions. As the curvature of a portion to be examined is greater, the degree of the decrease in the S/N is greater.

The present inventors have found the above problem and have conceived of a novel biological information measuring apparatus and a novel optical element.

The present disclosure includes a biological information measuring apparatus and an optical element set forth in the following items.

### Item 1

A biological information measuring apparatus according to Item 1 of the present disclosure includes
a light source that emits emission light with which a portion to be examined is to be irradiated;
a photodetector that detects light returning from the portion to be examined, the light being produced when the portion to be examined is irradiated with the emission light; and
an optical element that includes at least one lens, the optical element being disposed in an optical path between the light source and the portion to be examined.
At least one value selected from the group consisting of a thickness of the at least one lens and a refractive index of the at least one lens varies along a first direction extending from a center portion toward an outer edge portion, the center portion being a portion that includes a center of the at least one lens, and
the at least one value is locally minimum at the center portion and locally maximum at a first portion that lies between the center portion and the outer edge portion.

### Item 2

In the biological information measuring apparatus according to Item 1,
the at least one value may monotonically increase from the center portion toward the first portion and monotonically decrease from the first portion toward the outer edge portion.

### Item 3

In the biological information measuring apparatus according to Item 1 or 2,
the at least one lens may include a plurality of first portions, each of the plurality of first portions being the first portion,
one of the plurality of first portions may be present between the center portion and each of a plurality of points on the outer edge portion, and
a locus obtained by connecting the plurality of first portions may have a shape of one of a circle, an ellipse, and a rhombus.

### Item 4

In the biological information measuring apparatus according to Item 1 or 2,
the at least one value may be constant in a second direction orthogonal to both the first direction and a thickness direction of the at least one lens.

### Item 5

In the biological information measuring apparatus according to any one of Items 1 to 4,
the at least one lens may include a concave-convex surface that is concave at the center portion and convex at the first portion, and
the emission light incident on the at least one lens from the light source may exit through the concave-convex surface.

### Item 6

In the biological information measuring apparatus according to any one of Items 1 to 5,
a diffuser disposed in an optical path between the light source and the optical element may further be provided.

### Item 7

In the biological information measuring apparatus according to any one of Items 1 to 6,
the at least one lens may have a shape that is rotationally symmetric about an axis passing through the center, and
a sag amount may be expressed as a function of r including a term

α₁r²+α₂r⁴,
the sag amount indicating a difference between the thickness of the at least one lens at the center and the thickness of the at least one lens at a position that is spaced apart from the center by a distance r along a plane perpendicular to the axis,
where α₁ is a positive real number and α₂ is a negative real number.

### Item 8

In the biological information measuring apparatus according to any one of Items 1 to 6,
a sag amount may be expressed as a function of x and y including a term

α₁ₓx²+α_{iy}y²+α₂ₓx⁴+α_{2y}y⁴,
the sag amount indicating a difference between the thickness of the at least one lens at the center and the thickness of the at least one lens at a position that is spaced apart from the center by a distance x in the first direction and by a distance y in a second direction orthogonal to both the first direction and a thickness direction of the at least one lens,
where α₁ₓ and α_{1y} are positive real numbers and α₂ₓ and α_{2y} are negative real numbers.

### Item 9

In the biological information measuring apparatus according to any one of Items 1 to 6,
a sag amount may be expressed as a function of x including a term

α₁ₓx²+α₂ₓx⁴ or
as a function of y including a term

α_{1y}y²+α_{2y}y⁴,
the sag amount indicating a difference between the thickness of the at least one lens at the center and the thickness of the at least one lens at a position that is spaced apart from the center by a distance x in the first direction and by a distance y in a second direction orthogonal to both the first direction and a thickness direction of the at least one lens,
where α₁ₓ and α_{1y} are positive real numbers and α₂ₓ and α_{2y} are negative real numbers.

### Item 10

In the biological information measuring apparatus according to any one of Items 1 to 9,
the at least one lens may include a plurality of lenses arrayed two-dimensionally along a plane intersecting a center axis of the emission light,
at least one value selected from the group consisting of a thickness of each of the plurality of lenses and a refractive index of each of the plurality of lenses may vary along a direction extending from a center portion of each of the plurality of lenses toward an outer edge portion, the center portion being a portion that includes a center of each of the plurality of lens, and
the at least one value selected from the group consisting of the thickness of each of the plurality of lenses and the refractive index of each of the plurality of lenses may be locally minimum at the center portion and locally maximum at a portion that lies between the center portion and the outer edge portion.

### Item 11

The biological information measuring apparatus according to any one of Items 1 to 10 may further include
a control circuit, and
the control circuit may
   control the light source and the photodetector and
   generate information pertaining to a blood flow in the portion to be examined based on a signal indicating a quantity of the light detected by the photodetector.

### Item 12

In the biological information measuring apparatus according to any one of Items 1 to 11,
the emission light may be incident on the at least one lens as divergent light.

### Item 13

An optical element according to Item 13 of the present disclosure includes
at least one lens.
At least one value selected from the group consisting of a thickness of the at least one lens and a refractive index of the at least one lens varies along a first direction extending from a center portion toward an outer edge portion, the center portion being a portion that includes a center of the at least one lens, and
the at least one value is locally minimum at the center portion and locally maximum at a first portion that lies between the center portion and the outer edge portion.

### Item 14

In the optical element according to Item 13,
the at least one value may monotonically increase from the center portion toward the first portion and monotonically decrease from the first portion toward the outer edge portion.

### Item 15

In the optical element according to Item 13 or 14,
the at least one lens may include a plurality of first portions, each of the plurality of first portions being the first portion,
one of the plurality of first portions may be present between the center portion and each of a plurality of points on the outer edge portion, and
a locus obtained by connecting the plurality of first portions may have a shape of one of a circle, an ellipse, and a rhombus.

### Item 16

In the optical element according to any one of Items 13 to 15,
the at least one value may be constant in a second direction orthogonal to both the first direction and a thickness direction of the at least one lens.

### Item 17

In the optical element according to any one of Items 13 to 16,
the at least one lens may include a concave-convex surface that is concave at the center portion and convex at the first portion, and
the concave-convex surface may be disposed at a position where light exits from the at least one lens.

### Item 18

In the optical element according to any one of Items 13 to 17,
the at least one lens may have a shape that is rotationally symmetric about an axis passing through the center, and
a sag amount may be expressed as a function of r including a term

α₁r²+α₂r,
the sag amount indicating a difference between the thickness of the at least one lens at the center and the thickness of the at least one lens at a position that is spaced apart from the center by a distance r along a plane perpendicular to the axis,
where α₁ is a positive real number and α₂ is a negative real number.

### Item 19

In the optical element according to any one of Items 13 to 17,
a sag amount may be expressed as a function of x and y including a term

α₁ₓx²+α_{1y}y²+α₂ₓx⁴+α_{2y}y⁴,
the sag amount indicating a difference between the thickness of the at least one lens at the center and the thickness of the at least one lens at a position that is spaced apart from the center by a distance x in the first direction and by a distance y in a second direction orthogonal to both the first direction and a thickness direction of the at least one lens,
where α₁ₓ and α_{1y} are positive real numbers and α₂ₓ and α_{2y} are negative real numbers.

### Item 20

In the optical element according to any one of Items 13 to 17,
a sag amount may be expressed as a function of x including a term

α₁ₓx²+α₂ₓx⁴ or

as a function of y including a term

α_{1y}y²+α_{2y}y⁴,
the sag amount indicating a difference between the thickness of the at least one lens at the center and the thickness of the at least one lens at a position that is spaced apart from the center by a distance x in the first direction and by a distance y in a second direction orthogonal to both the first direction and a thickness direction of the at least one lens,
where α₁ₓ and α_{1y} are positive real numbers and α₂ₓ and α_{2y} are negative real numbers.

### Item 21

In the optical element according to any one of Items 13 to 20,
the at least one lens may include a plurality of lenses arrayed two-dimensionally along a plane intersecting a center axis of emission light,
at least one value selected from the group consisting of a thickness of each of the plurality of lenses and a refractive index of each of the plurality of lenses may vary along a direction extending from a center portion of each of the plurality of lenses toward an outer edge portion, the center portion being a portion that includes a center of each of the plurality of lens, and
the at least one value selected from the group consisting of the thickness of each of the plurality of lenses and the refractive index of each of the plurality of lenses may be locally minimum at the center portion and locally maximum at a portion that lies between the center portion and the outer edge portion.

### Item 22

An illumination apparatus according to Item 22 of the present disclosure includes
a light source that emits emission light with which an object is to be irradiated; and
an optical element.
The optical element includes at least one lens.
At least one value selected from the group consisting of a thickness of the at least one lens and a refractive index of the at least one lens varies along a first direction extending from a center portion toward an outer edge portion, the center portion being a portion that includes a center of the at least one lens.
The at least one value is locally minimum at the center portion and locally maximum at a first portion that lies between the center portion and the outer edge portion.
The at least one lens includes a concave-convex surface that is concave at the center portion and convex at the first portion, and
the emission light incident on the at least one lens from the light source exits through the concave-convex surface.

### Item 23

The illumination apparatus according to Item 22 may further include
a diffuser disposed in an optical path between the light source and the optical element.

In the present disclosure, all or a part of any of circuit, unit, device, part or portion, or any of functional blocks in the block diagrams may be implemented as one or more of electronic circuits including, but not limited to, a semiconductor device, a semiconductor integrated circuit (IC) or an LSI. The LSI or IC can be integrated into one chip, or also can be a combination of plural chips. For example, functional blocks other than a memory may be integrated into one chip. The name used here is LSI or IC, but it may also be called system LSI, VLSI (very large scale integration), or ULSI (ultra large scale integration) depending on the degree of integration. A Field Programmable Gate Array (FPGA) that can be programmed after manufacturing an LSI or a reconfigurable logic device that allows reconfiguration of the connection or setup of circuit cells inside the LSI can be used for the same purpose.

Further, it is also possible that all or a part of the functions or operations of the circuit, unit, device, part or portion are implemented by executing software. In such a case, the software is recorded on one or more non-transitory recording media such as a ROM, an optical disk or a hard disk drive, and when the software is executed by a processor, the software causes the processor together with peripheral devices to execute the functions specified in the software. A system or apparatus may include such one or more non-transitory recording media on which the software is recorded and a processor together with necessary hardware devices such as an interface.

Hereinafter, embodiments of the present disclosure will be described in more concrete terms. However, descriptions that are more elaborate than necessary may not be provided. For example, features that are already well known may not be described in detail, or duplicate descriptions of substantially identical configurations may be omitted. This is for preventing the following descriptions from becoming more lengthy than necessary and for facilitating understanding of a person skilled in the art. It is to be noted that the present inventors provide the appended drawings and the following descriptions to help a person skilled in the art understand the present disclosure at a sufficient level, and these drawings and descriptions are not intended to limit the subject set forth in the claims. In the following descriptions, identical or similar constituent elements are given identical reference characters.

Hereinafter, embodiments will be described with reference to the drawings.

### FIRST EMBODIMENT

First, a biological information measuring apparatus and an optical element according to a first embodiment of the present disclosure will be described.

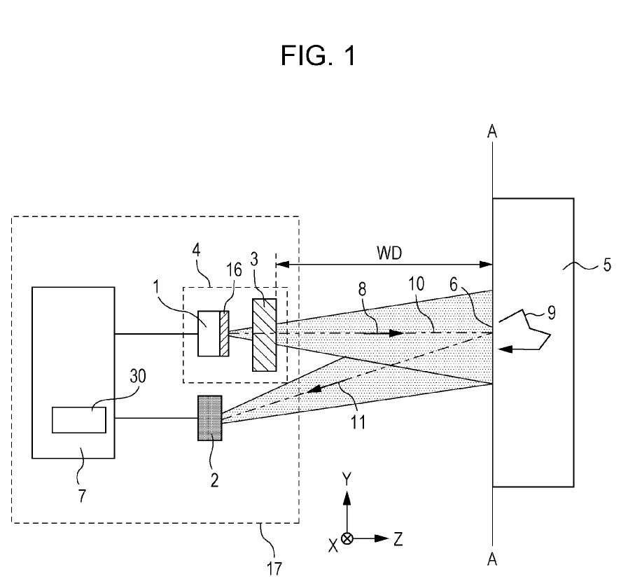

FIG. 1 is a schematic diagram for describing a configuration of the biological information measuring apparatus and how biological information is measured according to the first embodiment of the present disclosure.

A biological information measuring apparatus 17 according to the first embodiment includes a light source 1, a photodetector 2, a control circuit 7, and an optical element 3. The light source 1 emits emission light with which a portion 6 to be examined is irradiated. The photodetector 2 detects light that has been emitted from the light source 1 and reflected by the portion 6 to be examined. The optical element 3 is disposed in an optical path between the light source 1 and the portion 6 to be examined. The optical element 3 includes at least one lens. The control circuit 7 controls the light source 1 and the photodetector 2.

The control circuit 7 according to the present embodiment includes a signal processing circuit 30 that processes an electric signal (hereinafter, simply referred to as a signal) output from the photodetector 2. The signal processing circuit 30 generates information pertaining to the blood flow in the portion 6 to be examined on the basis of a signal indicating the quantity of light detected by the photodetector 2. The signal processing circuit 30 is used while being connected to the photodetector 2.

The control circuit 7 may, for example, be an integrated circuit that includes a processor, such as a central processing unit (CPU), and a memory. The control circuit 7, for example, executes a program recorded in the memory to thus cause the light source 1 to emit light and, in synchronization therewith, to cause the photodetector 2 to detect light.

The signal processing circuit 30 may be implemented, for example, by a digital signal processor (DSP), a programmable logic device (PLD) such as a field programmable gate array (FPGA), or a combination of a central processing unit (CPU) or a graphics processing unit (GPU) and a computer program. The control circuit 7 and the signal processing circuit 30 may be a single integrated circuit or may be individual separate circuits.

The biological information measuring apparatus 17 according to the present embodiment includes the control circuit 7. Alternatively, the control circuit 7 may be an element external to the biological information measuring apparatus 17.

The light source 1 may, for example, be a laser light source, such as a laser diode (LD), that successively emits pulsed light or a light emitting diode (LED). The light source 1 can start or stop emitting light and can change the emission power in accordance with an instruction from the control circuit 7 and can thus generate generally desired pulsed light.

The light source 1 emits light at a wavelength of no shorter than 650 nm nor longer than 950 nm, for example. This wavelength range is within a wavelength range of from red light to near-infrared rays. The above wavelength range is referred to as the biological window and is known for its low absorptance within a body. The descriptions are based on the assumption that the light source 1 according to the present embodiment emits light in the above wavelength range, but light in other wavelength ranges may also be used. In the present specification, the term “light” is used not only for visible light but also for infrared rays.

In the present embodiment, a diffuser 16 is disposed in an optical path between the light source 1 and the optical element 3 at a position in proximity to the light source 1. The diffuser 16 broadens the radiation angle of the emission light with a Gaussian distribution emitted from the light source 1 to transform the emission light to light with a Lambertian distribution, for example. The light diffused by the diffuser 16 is incident on the optical element 3. The optical element 3 further transforms the optical intensity distribution of light 8 to increase the optical intensity in the peripheral portion. The light transformed by the optical element 3 hits a surface (A-A plane) of the portion 6 to be examined that is spaced apart from the optical element 3 by a distance WD. The diffuser 16 is not an essential constituent element. However, providing the diffuser 16 renders it easier to design and manufacture the optical element 3.

The light source 1 including the diffuser 16 may be integrated with the optical element 3 to form a light source module 4. This renders it even easier to handle the light source 1 and the optical element 3.

In the following descriptions, “scattered light” includes reflected scattered light and transmitted scattered light. The reflected scattered light may simply be referred to as “reflected light.”

An organism is a scattering body. When a subject 5 is an organism, some of the light 8 incident on the portion 6 to be examined returns to the biological information measuring apparatus 17 as direct reflected light. The remaining portion of the light enters into the subject 5 through the skin on the surface of the portion 6 to be examined and is diffused, while some of that light is absorbed by the subject 5, to result in internal scatted light 9. Reflected scattered light 11 that has emerged from the inside of the subject 5 is detected by the photodetector 2. Incident on the photodetector 2 are direct reflected light that results in intense noise light and the reflected scattered light 11 having information on the internal blood flow. The optical path length of the reflected scattered light 11 is greater than the optical path length of the direct reflected light. Therefore, the arrival time of the reflected scattered light 11 at the photodetector 2 is later than the arrival time of the direct reflected light at the photodetector 2. Detecting light at a timing later than the time when the direct reflected light arrives at the photodetector 2 makes it possible to reduce the component of the direct reflected light, namely, the noise light component to be included in a detection signal and to increase the proportion of the component of the reflected scattered light 11.

The reflected scattered light 11 from the inside of the organism is light that has been transmitted through a blood vessel and so on and thus includes biological information on the heartbeat, the blood flow volume, the blood pressure, the saturation of peripheral oxygen, and so on. These pieces of biological information can be used for a variety of purposes including a medical examination and an estimation of an emotional state.

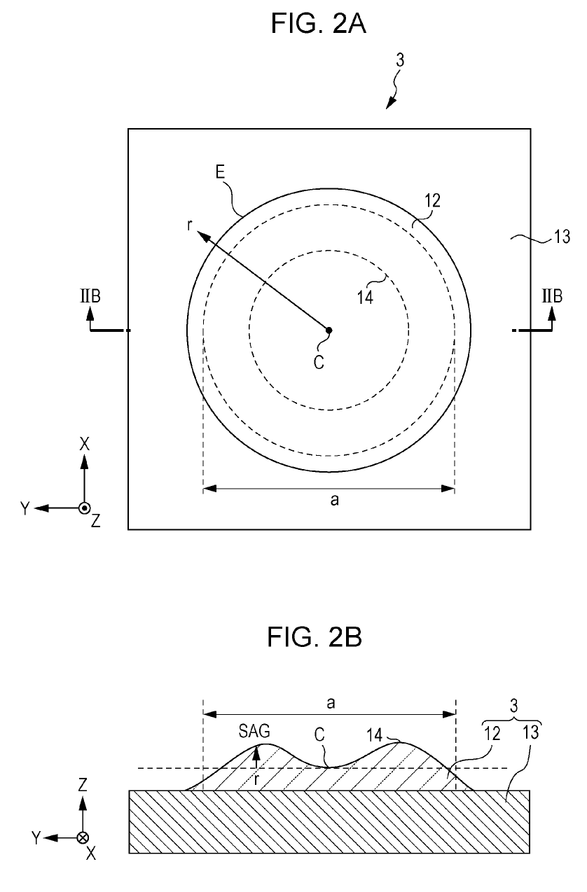

FIG. 2A is a plan view schematically illustrating a configuration of the optical element 3 according to the first embodiment of the present disclosure. FIG. 2B is a sectional view taken along the IIB-IIB line indicated in FIG. 2A.

The optical element 3 according to the present embodiment includes a substrate 13 and a lens 12 provided at a center portion of the substrate 13. The lens 12 has a circular shape as viewed in the optical axis direction, or the Z-direction. The lens 12 has a shape that is rotationally symmetric about an axis that passes through the center of the lens 12, or the optical axis. The thickness of the lens 12 varies along a first direction, and the first direction extends from a center portion C of the lens 12 toward an outer edge portion E. The thickness of the lens 12 is locally minimum at the center portion C and locally maximum at a first portion (hereinafter, may be referred to as a “local maximum portion”) that lies between the center portion C and the outer edge portion E. To be more specific, the first portion at which the thickness of the lens 12 is locally maximum, or the local maximum portion, is present at each of a plurality of points that lie between the center portion C of the lens 12 and a plurality of points on the outer periphery, or the outer edge portion, of the lens 12. FIG. 2A illustrates a locus 14 obtained by connecting these first portions. In the present embodiment, the locus 14 is circular. As illustrated in FIG. 2B, the thickness of the lens 12 monotonically increases from the center portion C toward the local maximum portion and monotonically decreases from the local maximum portion toward the outer edge portion E.

In the present embodiment, the thickness of the lens 12 varies in accordance with the distance from the center portion C. Instead of the thickness of the lens 12 or in addition to the thickness of the lens 12, the refractive index of the lens 12 may be varied in a similar manner. In other words, it suffices that at least one of the thickness and the refractive index of the lens 12 in the optical element 3 vary in a direction extending from the center portion C of the lens 12 toward the outer edge portion E. The lens 12 is designed such that at least one of the thickness and the refractive index of the lens 12 is locally minimum at the center portion C and locally maximum at the first portions that lie between the center portion C and the outer edge portion E. The use of such a lens 12 makes it possible to collect more light onto a peripheral portion.

The center portion C coincides with the optical axis center in the present embodiment, and the center axis of the emission light from the light source 1 coincides with this optical axis center. Therefore, the center portion C may be referred to as the optical axis center in the present embodiment. The portion at which the thickness of the lens 12 is locally minimum need not strictly coincide with the optical axis center. It suffices that the thickness or the refractive index be locally minimum at a point in the vicinity of the optical axis center and that the thickness or the refractive index be locally maximum at points outside the aforementioned point. In the present specification, the term “center portion” refers to a point within a region with some spread containing the optical axis center and therearound.

The substrate 13 and the lens 12 may be fabricated from the same material, such as a cycloolefin resin (e.g., ZEONEX (registered trademark) available from Zeon Corporation). Alternatively, the substrate 13 and the lens 12 may be formed of different materials.

The substrate 13 and the lens 12 may be formed of a material that is substantially transparent to the light from the light source 1. The substrate 13 and the lens 12 can also be formed of a resin other than a cycloolefin resin, such as polycarbonate, a polymethyl methacrylate resin (PMMA), a norbornene resin (e.g., ARTON (registered trademark) available from JSR Corporation), or glass. The use of a resin can render it easier to integrally form the substrate 13 and the lens 12 from the same material.

The shape of the optical element 3 or the lens 12 can be expressed by a distribution of the sag amount indicating the difference between the thickness of the lens 12 at each point and the thickness of the lens 12 at the center portion C. The sag amount of the optical element 3 according to the present embodiment has a local minimum value at the center portion C, or the optical axis center, and has a local maximum value at a portion other than the center portion C. This local maximum value is also the global maximum value. The lens 12 in the optical element 3 has a structure that is rotationally symmetric about the center portion C, or the optical axis center. When loci obtained by connecting the portions of the lens 12 at which the lens 12 has the same thickness are projected onto an XY-plane, the loci result in concentric circles with their centers lying at the center portion C.

Hereinafter, a physical action of the optical element 3 according to the present embodiment will be described. The lens 12 in the optical element 3 has an effective diameter a. The lens 12 has a concave lens shape around the center portion and a convex lens shape at the peripheral portion. Thus, the optical element 3 disperses light, of the light emitted from the light source 1, incident on the center portion and therearound and condenses light incident on the peripheral portion. In other words, the optical element 3 functions to make the intensity of the light 8 higher at the peripheral portion than at the center portion and therearound in the A-A plane where the portion 6 to be examined is present.

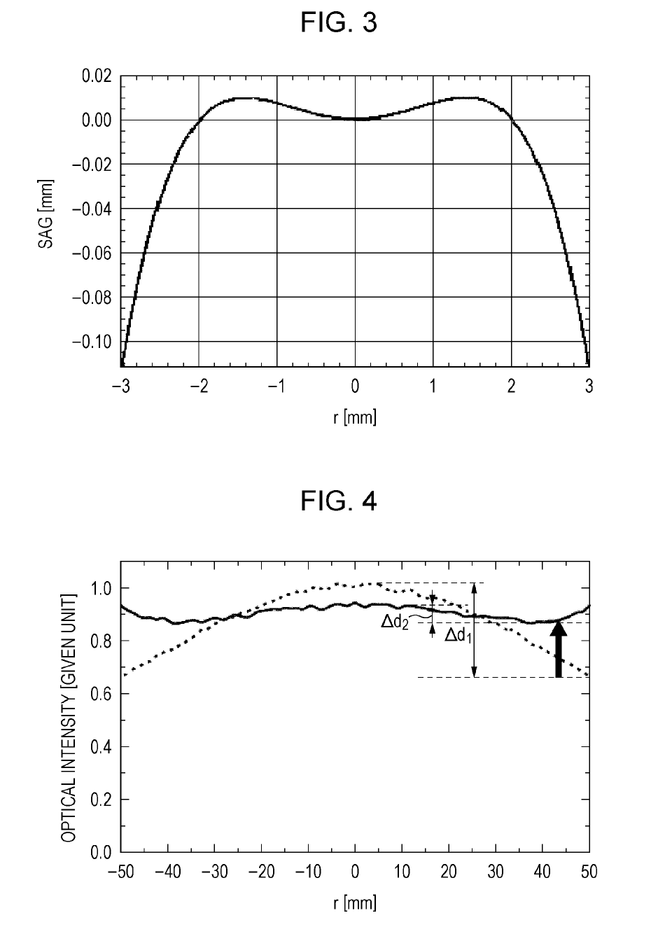

FIG. 3 is a graph illustrating an example of a relationship between the sag amount of the optical element 3 and the radius r from the optical axis center according to the first embodiment of the present disclosure. The sag amount takes a local minimum value at the center portion C and a local maximum value at the peripheral portion. In this example, the local maximum value coincides with the global maximum value.

The present inventors have found that the optical element 3 having such a sag amount distribution can be treated as an even-order aspherical lens. Furthermore, the present inventors have found that the sag amount can be expressed as a function of r including the term α₁r²+α₂r⁴, in which r is the distance from the center portion C, or the optical axis center, of the optical element 3, α₁ is a positive real number, and α₂ is a negative real number. Here, |α₁|>|α₂| holds. Describing with the use of the xy-coordinates yields r²=x²+y², and thus the aforementioned sag amount can be expressed as a function of x and y including the term α₁(x²+y²)+α₂(x²+y²)²=α₁x²+α₂x⁴+α₁y²+α₂y⁴+2α₂x²y².

It is also possible to employ a design method in which the optical element 3 is treated as a lens other than an even-order aspherical lens. For example, a design technique for a refractive optical element having an odd-order aspherical surface, a toroidal surface, a Zernike standard sag surface, or the like may be employed. Furthermore, a design technique for a diffractive optical element having a hologram surface, a grating surface, or the like can also be employed. Regardless of with which technique the optical element 3 is designed, it suffices that the optical element 3 have a shape or a function of a concave lens and a convex lens at the center portion C and the peripheral region, respectively, of the lens 12. As long as the optical element 3 is designed in this manner, the optical element 3 can provide an advantageous effect equivalent to the advantageous effect of the present embodiment in which the optical element 3 increases the intensity of the light 8 at the peripheral portion.

As an example, the distance WD between the optical element 3 and the portion 6 to be examined is set to 100 mm, the distance from the surface of the diffuser 16 to the optical element 3 is set to 2 mm, the thickness of the substrate 13 is set to 3 mm, the sizes of the portion 6 to be examined in the X-direction and the Y-direction are each set to 100 mm, and the effective diameter a of the lens 12 is set to 4.24 mm. Then, when α₁=0.01 and α₂=−0.0025, the sag amount of the lens 12 can be expressed as a function of r including the term α₁r²+α₂r⁴, or in this case, expressed by α₁r²+α₂r⁴, for example. When r=1.41 mm, the local maximum value (global maximum value) of the sag amount is 10.1 μm.

FIG. 4 is a diagram illustrating the intensity distribution (solid line) of the emission light from the biological information measuring apparatus 17 toward the portion 6 to be examined along a plane perpendicular to the optical axis according to the first embodiment of the present disclosure and the intensity distribution (dotted line) of the emission light along a plane perpendicular to the optical axis obtained when the optical element 3 is not provided.

As indicated by the dotted line in FIG. 4, when the optical element 3 is not provided, obtained is an optical intensity distribution of a Lambertian distribution in which the optical intensity is reduced at the peripheral portion. In this case, the difference Δd₁ between the highest value and the lowest value of the intensity of the light incident on the A-A plane is large. In contrast, as indicated by the solid line, when the optical element 3 is provided, the difference Δd₂ between the highest value and the lowest value of the intensity of the light incident on the A-A plane is smaller than the difference Δd₁. In other words, Δd₂<Δd₁ holds. The use of the optical element 3 makes it possible to increase the optical intensity at the peripheral portion and to bring the optical intensity distribution on the portion 6 to be examined closer to a uniform distribution. Accordingly, the S/N of a detection signal of the reflected scattered light 11 from the peripheral region of the portion 6 to be examined can be improved.

Next, a biological information measuring apparatus and an optical element according to a modification of the first embodiment of the present disclosure will be described.

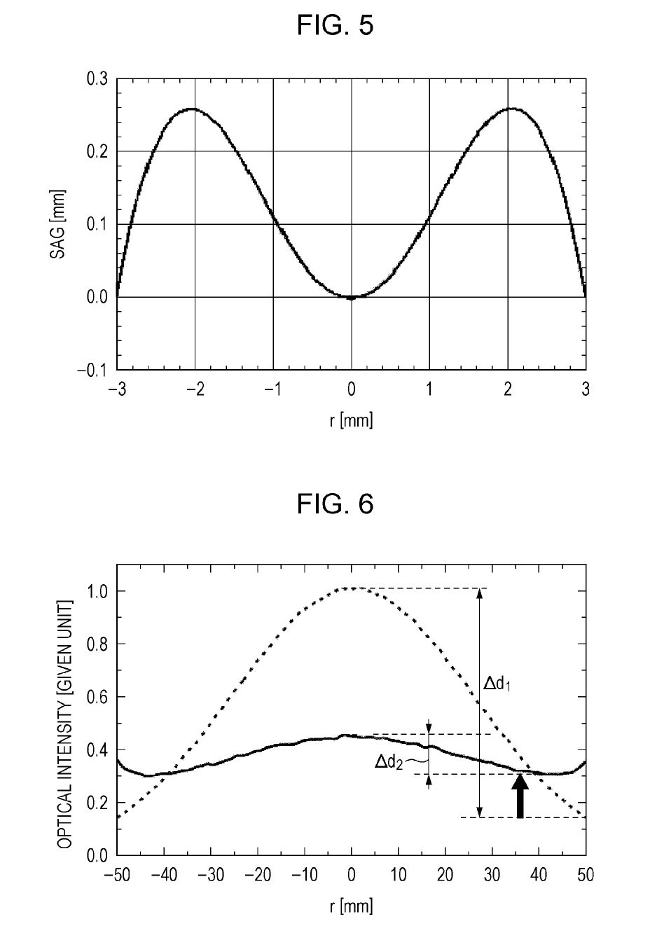

FIG. 5 is a graph illustrating a relationship between the sag amount of the optical element and the radius r from the optical axis center according to the modification of the first embodiment of the present disclosure.

A difference from the biological information measuring apparatus according to the first embodiment described above is that the diffuser 16 is not disposed in an optical path between the light source 1 and the optical element 3 and the optical element 3 has a structure that functions as a diffuser as well. The light source 1 and the optical element 3 is integrated into the light source module 4.

The optical element 3 transforms the light 8 of the Gaussian distribution emitted from the light source 1 such that the intensity of the light at the peripheral portion is increased by the optical element 3. The transformed light 8 hits the portion 6 to be examined in the A-A plane. In this case, it has been found that, with a real number α₃ being added, the sag amount can be expressed as a function of r including the term α₁r²+α₂r⁴+α₃r⁶.

As an example, the distance WD between the optical element 3 and the portion 6 to be examined is set to 100 mm, the thickness of the substrate 13 is set to 3 mm, the sizes of the portion 6 to be examined in the X-direction and the Y-direction are each set to 100 mm, and the effective diameter a of the lens 12 is set to 4.24 mm. Then, when α₁=0.13, α₂=−0.018, and α₃=0.004; the sag amount of the lens 12 can be expressed as a function of r including the term α₁r²+α₂r⁴+α₃r⁶, or in this case, expressed by α₁r²+α₂r⁴+α₃r⁶, for example. When r=2.05 mm, the local maximum value (global maximum value) of the sag amount is 258 μm.

In this modification, the optical element 3 functions as a diffuser as well. Therefore, as compared to the optical element according to the first embodiment, |α₁|, |α₂|, and the maximum sag amount increase. In this example, |α₁|>|α₂|>|α₃| holds.

FIG. 6 is a diagram illustrating the intensity distribution (solid line) of the emission light from the biological information measuring apparatus 17 toward the portion 6 to be examined according to the modification of the first embodiment of the present disclosure and the intensity distribution (dotted line) of the emission light obtained when the optical element 3 is not provided.

As indicated by the dotted line in FIG. 6, when the optical element 3 is not provided, obtained is an optical intensity distribution of a Lambertian distribution in which the optical intensity is reduced at the peripheral portion. In this case, the difference Δd₁ between the highest value and the lowest value of the intensity of the light incident on the A-A plane is large. In contrast, as indicated by the solid line, when the optical element 3 according to the modification is provided, the difference Δd₂ between the highest value and the lowest value of the intensity of the light incident on the A-A plane is reduced to a great extent (Δd₂<Δd₁), and the optical intensity at the peripheral portion can be improved. Accordingly, the S/N of a detection signal of the reflected scattered light 11 from the peripheral region of the portion 6 to be examined can be improved in this modification as well.

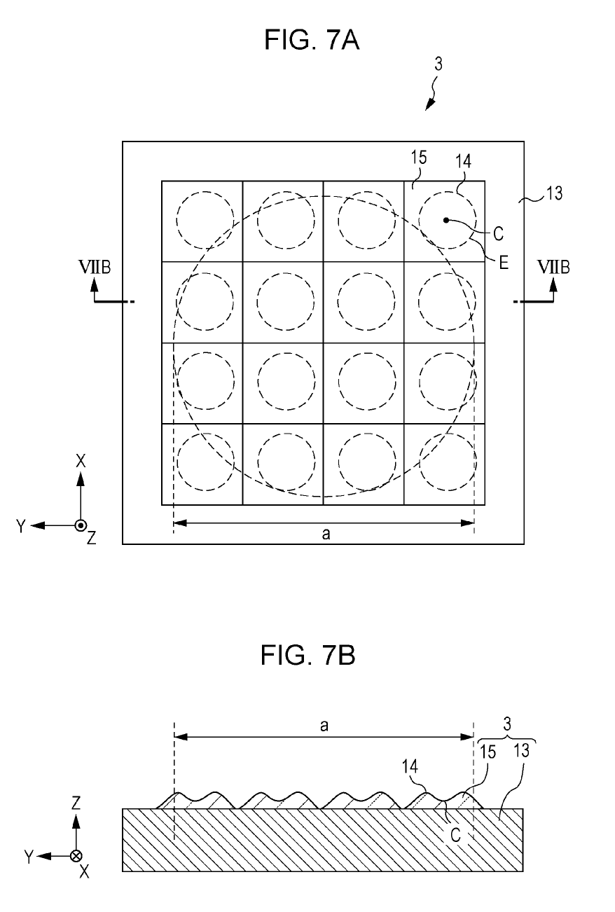

FIG. 7A is a plan view schematically illustrating a configuration of an optical element 3 according to another modification of the first embodiment of the present disclosure. FIG. 7B is a sectional view taken along the VIIB-VIIB line indicated in FIG. 7A.

The optical element 3 includes a lens array 15 that includes a plurality of lenses arrayed two-dimensionally along a plane intersecting the center axis of the light emitted from the light source 1. At least one of the thickness and the refractive index of each lens is locally minimum at the center portion C of each lens and locally maximum at a portion that lies between the center portion C and the outer edge portion E of each lens.

Hereinafter, an example in which the thickness of each lens varies in accordance with the distance from the center will be described, but the configuration may be such that the refractive index of each lens varies in accordance with the distance from the center.

The optical element 3 according to this modification includes the substrate 13 and the lens array 15 disposed on the substrate 13. The lens array 15 includes a plurality of lenses arrayed in the X-direction and the Y-direction. In the example illustrated in FIG. 7A, the lens array 15 includes 16 lenses arrayed in a 4 by 4 matrix, but the number of the lenses and how the lenses are arrayed may be determined as desired.

The sag amount of each lens has a local minimum value at the center portion C, or the optical axis center, and a local maximum value at a portion other than the center portion C. In this example as well, the locus 14 obtained by connecting the portions at which the sag amount of each lens is locally maximum has a circular shape. A difference from the optical element 3 according to the first embodiment is that the lens array 15 is used instead of a single lens. Thus, the tolerance in the positioning of the optical element 3 in the X- and Y-directions in which the lenses are arrayed improves. The sag amount of each lens can be expressed as a function of r including the term α₁r²+α₂r⁴, in a similar manner to the example illustrated in FIG. 2A and FIG. 2B. Here, |α₁|>|α₂| holds. Each lens in the optical element 3 has a concave lens shape and a convex lens shape at the center portion C and the peripheral region, respectively.

In the present modification, the lenses constituting the array all have the same structure. However, not all the lenses need to have the same structure. The structure of the lens array may be modified in accordance with the distribution of light from the light source 1.

### SECOND EMBODIMENT

Next, a biological information measuring apparatus according to a second embodiment of the present disclosure will be described. The following descriptions center on the differences from the biological information measuring apparatus according to the first embodiment described above.

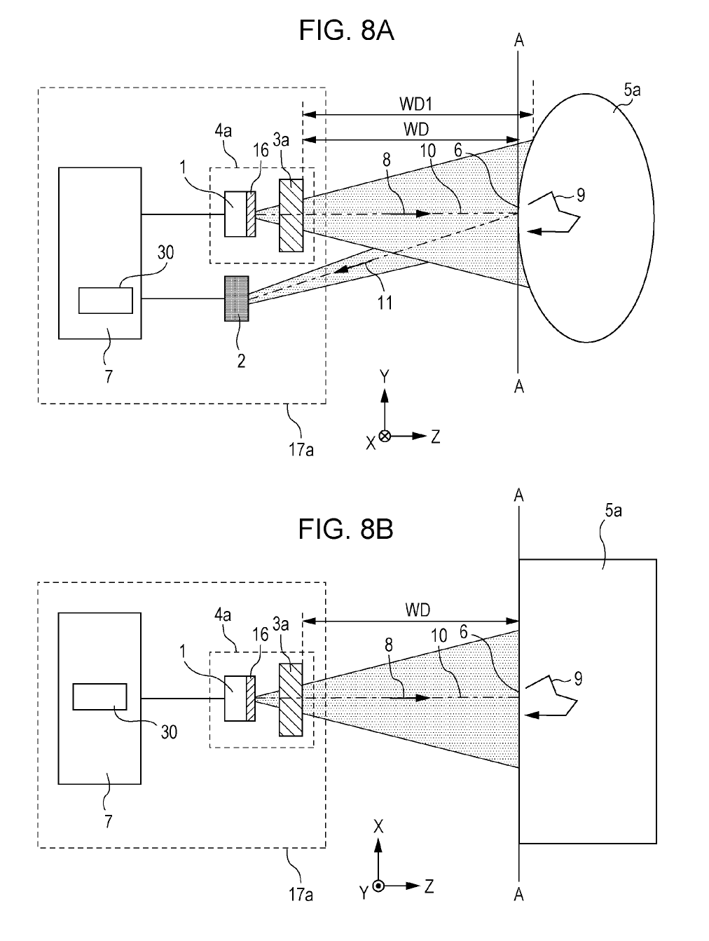

FIGS. 8A and 8B are schematic diagrams for describing a configuration of the biological information measuring apparatus and how biological information is measured according to the second embodiment of the present disclosure. FIG. 8A illustrates a configuration of the biological information measuring apparatus as viewed in the X-direction. FIG. 8B illustrates a configuration of the biological information measuring apparatus as viewed in the Y-direction. The light source 1 and an optical element 3a is integrated into a light source module 4a.

A subject 5a according to the present embodiment is, for example, a portion of an organism having a shape that can be approximated to an cylindroid, such as an arm or a leg. As illustrated in FIG. 8A, the portion 6 to be examined has a curved shape along a YZ-section and its curvature is relatively large. In contrast, as illustrated in FIG. 8B, the portion 6 to be examined does not have a curved shape along an XZ-section.

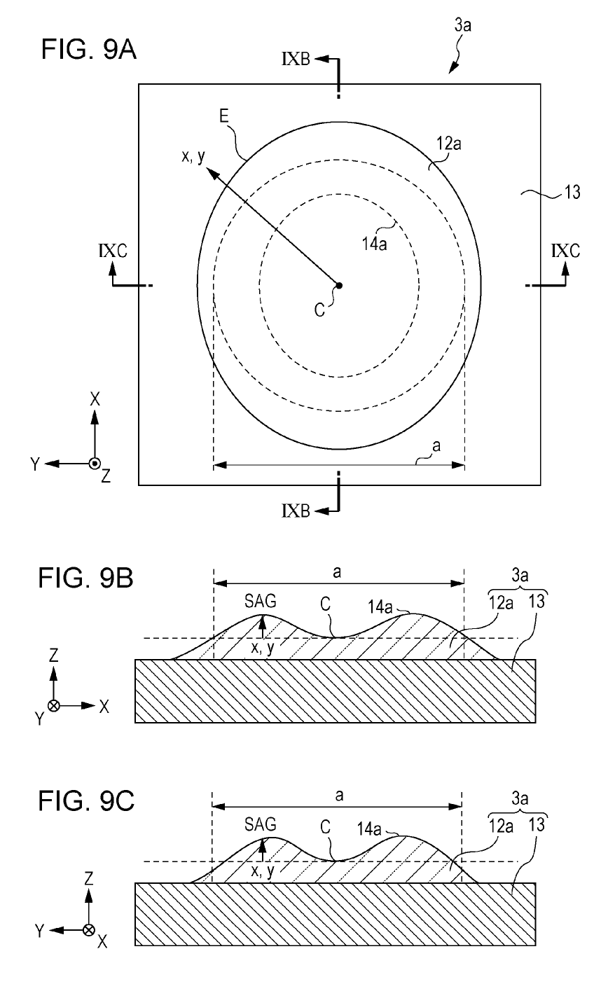

FIG. 9A is a plan view schematically illustrating a configuration of the optical element according to the second embodiment of the present disclosure. FIG. 9B is a sectional view taken along the IXB-IXB line indicated in FIG. 9A. FIG. 9C is a sectional view taken along the IXC-IXC line indicated in FIG. 9A.

When the curvature of the portion 6 to be examined differs in the X-direction and in the Y-direction, an optimal sag amount distribution is obtained for each of the directions, and the obtained results are combined. Then, an optimal optical element 3a can be designed. It has been found that the loci obtained by connecting the portions at which the sag amount has an identical value in the sag amount distribution obtained as described above result in concentric ellipses.

In the present embodiment as well, the sag amount of a lens 12a in the optical element 3a has a local minimum value at the center portion C, or the optical axis center, and a local maximum value at a portion other than the center portion C. The shape of a locus 14a obtained by connecting the portions at which the sag amount of the lens 12a is locally maximum is approximately an ellipse having a major axis extending in the X-direction and a minor axis extending in the Y-direction. It has been found that the use of the lens 12a having a minor axis extending in the Y-direction makes it possible to obtain an advantageous effect in that the intensity of the light 8 along the A-A plane is greater at the peripheral portions in the Y-direction than at the peripheral portions in the X-direction.

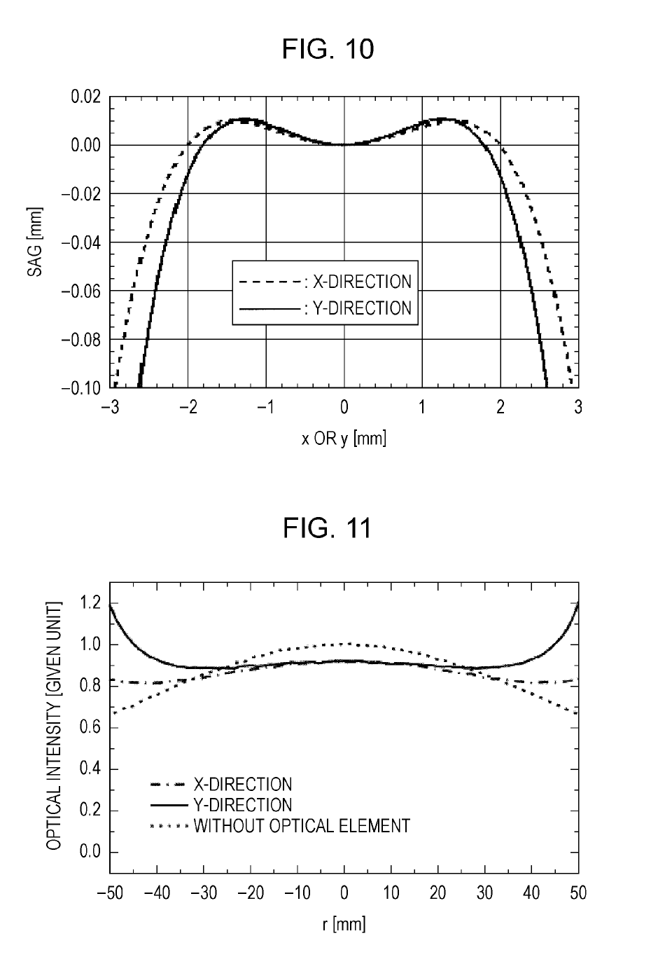

FIG. 10 is a graph illustrating a distribution of the sag amounts of the lens 12a in the X-direction and the Y-direction according to the second embodiment of the present disclosure.

The optical axis center of the element serves as the origin, α₁ₓ and α_{1y} are positive real numbers, and α₂ₓ and α_{2y} are negative real numbers. Then, the present inventors have found that the sag amount of the optical element 3a can be expressed as a function of x and y including the term α₁ₓx²+α₂ₓx⁴+α_{1y}y²+α_{2y}y⁴.

As an example, the distance WD between the optical element 3a and the center portion of the portion 6 to be examined is set to 100 mm, the distance from the surface of the diffuser 16 to the optical element 3a is set to 2 mm, the thickness of the substrate 13 is set to 3 mm, the sizes of the portion 6 to be examined in the X-direction and the Y-direction are each set to 100 mm, and the effective diameter a of the lens 12a is set to 4.24 mm. Then, when α_{1y}=0.013 and α_{2y}=−0.004, the sag amount in the Y-direction can be expressed as a function of y including the term α_{1y}y²+α_{2y}y⁴, or in this case, expressed by α_{1y}y²+α_{2y}y⁴, for example. When y=1.28 mm, the local maximum value (global maximum value) of the sag amount is 10.6 μm. When α₁ₓ=0.01 and α₂ₓ=−0.0025, the sag amount in the X-direction can be expressed as a function of x including the term α₁ₓx²+α₂ₓx⁴, or in this case, expressed by α₁ₓx²+α₂ₓx⁴, for example. When x=1.41 mm, the local maximum value (global maximum value) of the sag amount is 10.6 μm.

Therefore, the shape of the locus 14a is approximately an ellipse having a major axis extending in the X-direction. In this example, the eccentricity e is 0.43. When the sizes of the ellipse in the major axis direction and the minor axis direction are represented by Sₓ and S_{y}, respectively, the eccentricity is defined as e=(1−(S_{y}/Sₓ)²)^{0.5}.

FIG. 11 illustrates the intensity distribution (dashed-dotted line) in the X-direction and the intensity distribution (solid line) in the Y-direction of the emission light from the biological information measuring apparatus 17a toward the portion 6 to be examined along a plane perpendicular to the optical axis according to the second embodiment of the present disclosure and the intensity distribution (dotted line) of the emission light along a plane perpendicular to the optical axis obtained when the optical element 3a is not provided.

As indicated by the dotted line in FIG. 11, when the optical element 3a is not provided, obtained is an optical intensity distribution in which the optical intensity is reduced at the peripheral portion. The difference between the highest value and the lowest value of the optical intensity is large. However, as indicated by the dashed-dotted line and the solid line, the use of the optical element 3a makes it possible to increase the optical intensity at the peripheral portion and to bring the optical intensity distribution on the portion 6 to be examined closer to a more uniform distribution. Accordingly, the S/N of a detection signal of the reflected scattered light 11 from the peripheral region of the portion 6 to be examined can be improved.

The shape of the locus 14a obtained by connecting the portions at which the sag amount of the lens 12a is locally maximum is not limited to an ellipse.

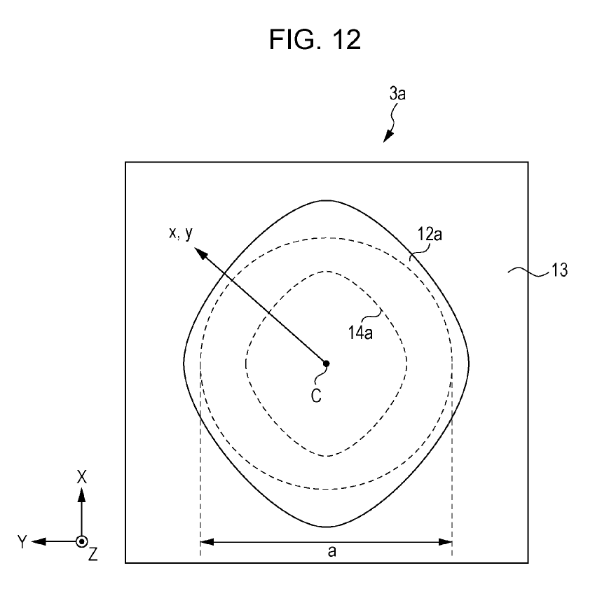

FIG. 12 is a plan view schematically illustrating another configuration of an optical element according to the second embodiment of the present disclosure. In the example illustrated in FIG. 12, the shape of the locus 14a obtained by connecting the portions at which the sag amount of the lens 12a is locally maximum is a rounded rhombus. Such a shape can be derived mathematically. In other words, the sag amount of the optical element 3a can be expressed as a function of x and y including the term α₁ₓx²+α₂ₓx⁴+α_{1y}y²+α_{2y}y⁴. The locus 14a is accurately derived from this function, and the shape of the locus 14a can be approximated to an ellipse as described above and can also be a rhombus or a rhombus with four rounded corners, for example, depending on the conditions.

Next, a biological information measuring apparatus and an optical element according to a modification of the second embodiment of the present disclosure will be described.

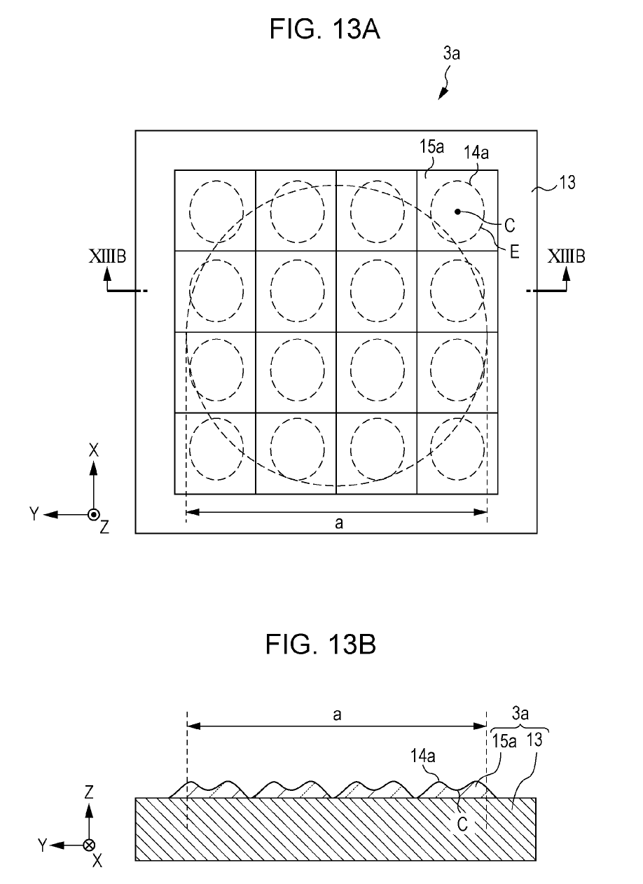

FIG. 13A is a plan view schematically illustrating a configuration of an optical element according to a modification of the second embodiment of the present disclosure. FIG. 13B is a sectional view taken along the XIIIB-XIIIB line indicated in FIG. 13A.

The optical element 3a according to the present modification includes the substrate 13 and a lens array 15a disposed on the substrate 13. The sag amount of each lens has a local minimum value at the center portion C, or the optical axis center, and a local maximum value at a portion other than the center portion C. The shape of the locus 14a obtained by connecting the local maximum portions in each lens is an ellipse. A difference from the optical element 3a according to the second embodiment is that the lens array 15a including a plurality of lenses is used instead of a single lens. Constituting the optical element 3a as an array improves the tolerance in the positioning of the optical element 3a in the X-direction and the Y-direction. The sag amount of the each lens can be expressed similarly as a function of x and y including α₁ₓx²+α₂ₓx⁴+α_{1y}y²+α_{2y}y⁴. In the above, |α₁ₓ|>|α₂ₓ| and |α_{1y}|>|α_{2y}| hold. The optical element 3a has a concave lens shape and a convex lens shape at the center portion C and therearound and at the peripheral region, respectively, of each lens.

Not all the lenses constituting the array need to have the same structure. The structure of each lens may be modified in accordance with the distribution of light from the light source 1. The shape of the locus 14a obtained by connecting the local maximum portions is not limited to an ellipse. The shape of the locus 14 or 14a may be a rhombus or a rhombus with four rounded corners, for example.

### Third Embodiment

Next, a biological information measuring apparatus according to a third embodiment of the present disclosure will be described. The following descriptions center on the differences from the biological information measuring apparatus according to the second embodiment described above.

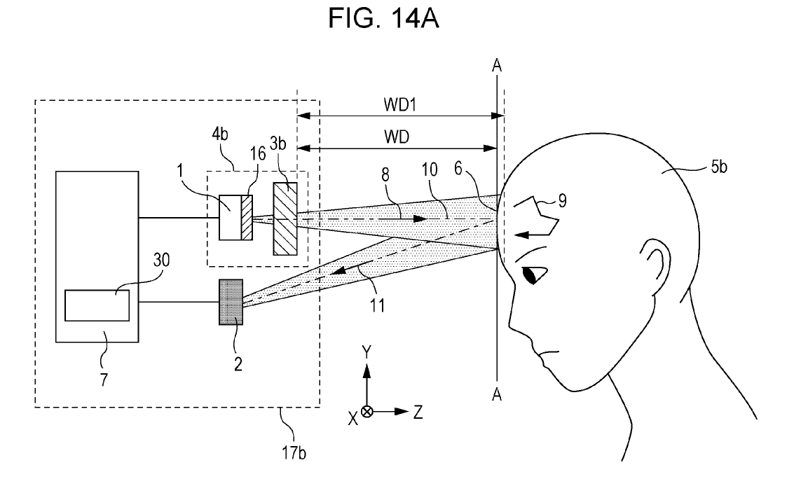

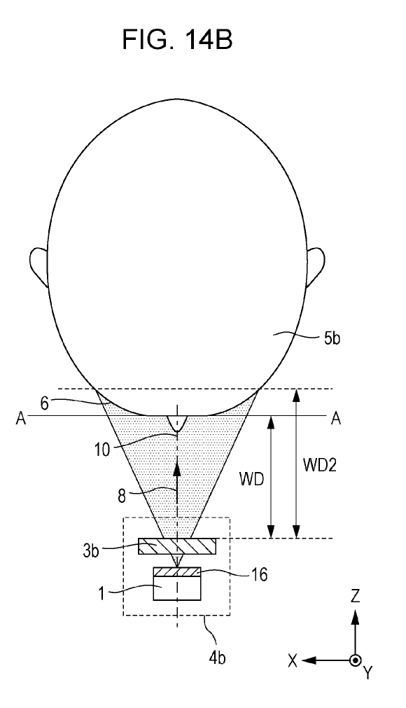

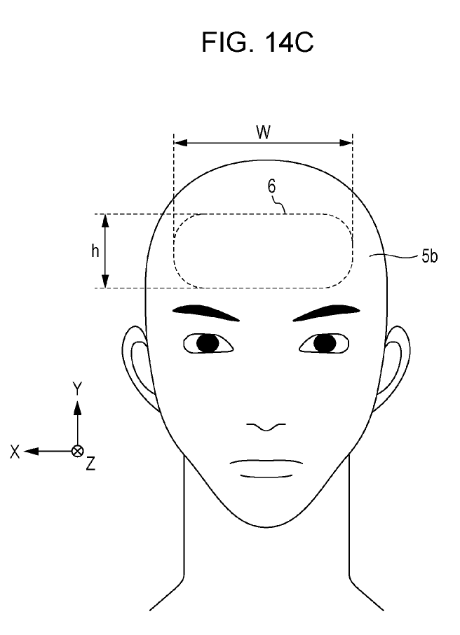

FIG. 14A to FIG. 14C are schematic diagrams for describing a configuration of a biological information measuring apparatus and how biological information is measured according to the third embodiment of the present disclosure. FIG. 14A illustrates a configuration of the biological information measuring apparatus as viewed in the X-direction (from the side). FIG. 14B illustrates a configuration of the biological information measuring apparatus as viewed in the Y-direction (from the above). FIG. 14C illustrates a portion to be examined as viewed in the Z-direction (from the front).

A biological information measuring apparatus 17b according to the third embodiment is, for example, an apparatus that contactlessly measures a brain function. The photodetector 2 is an image sensor having an electronic shutter function. The diffuser 16 transforms the divergent light with a Gaussian distribution emitted from the light source 1 to light with a Lambertian distribution, for example. An optical element 3b increases the intensity of the transformed light at the peripheral portion. The light 8 transmitted through the optical element 3b hits the portion 6 to be examined of a subject 5b. The portion 6 to be examined in the present embodiment is a forehead. The size of the portion 6 to be examined in the X-direction is designated by W, and the size in the Y-direction is designated by h. The light 8 may be continuous light but is pulsed light in the present embodiment. The light source 1 repeatedly emits pulsed light in accordance with the sensitivity of the photodetector 2. The pulse duration is, for example, no less than 0.1 ns nor more than 1 μs and is 11 ns in a certain example. The light source 1 and the optical element 3b are integrated into a light source module 4b.

Present inside the forehead serving as the portion 6 to be examined are, sequentially from the surface side, the scalp (thickness of approximately 3 mm to 6 mm), the cranial bone (thickness of approximately 5 mm to 10 mm), the cerebrospinal fluid layer (thickness of approximately 2 mm), and the brain tissue. The thickness ranges indicated in the parentheses represent individual differences. Some of the light 8 emitted toward the portion 6 to be examined returns to the biological information measuring apparatus 17b as direct reflected light. The remaining portion of the light results in the internal scatted light 9 that enters inside the portion 6 to be examined through the surface thereof. Some of the internal scatted light 9 is absorbed, and the remaining portion thereof is diffused and emerges from the surface of the portion 6 to be examined. The internal scatted light 9 that has entered inside the portion 6 to be examined includes information on the cerebral blood flow at a portion approximately 10 mm to 18 mm deep from the surface. The internal scatted light 9 that has emerged from the portion 6 to be examined returns to the biological information measuring apparatus 17b as the reflected scattered light 11 from the inside. Incident on the photodetector 2 are the direct reflected light that results in intense noise light and the reflected scattered light 11 having information on the cerebral blood flow. The optical path length of the reflected scattered light 11 is greater than the optical path length of the direct reflected light. Therefore, the arrival time of the reflected scattered light 11 at the photodetector 2 is later than the arrival time of the direct reflected light at the photodetector 2. Detecting the light with the use of the electronic shutter function of the image sensor at a timing later than the arrival time of the direct reflected light makes it possible to reduce the noise light and to improve the S/N of a signal pertaining to the cerebral blood flow.

The light source 1 is a multi-wavelength light source that emits light with at least two wavelengths. The light source 1 separately emits the light 8, which is pulsed light, at each wavelength in accordance with an instruction from the control circuit 7.

The light source 1 includes, for example, a structure in which a plurality of laser chips are embedded within a package. The two wavelengths to be used may be 750 nm and 850 nm, for example. The absorptance of light by oxidized hemoglobin and reduced hemoglobin varies in the wavelengths of 750 nm and 850 nm, for example. Therefore, carrying out a computation with a combination of two electric signals obtained by using the two respective wavelengths makes it possible to measure information pertaining to the cerebral blood flow in the portion 6 to be examined, such as the proportions of oxidized hemoglobin and reduced hemoglobin. This computation is executed by the signal processing circuit 30 (refer to FIG. 1).

When the portion 6 to be examined is a forehead region of a head portion of an organism, the amount of change in the cerebral blood flow in the frontal lobe or the amount of change in the oxidized hemoglobin concentration and the reduced hemoglobin concentration can be measured. On the basis of these pieces of information, information on the emotional state or the like can be sensed. For example, under the concentrated state, the cerebral blood flow volume increases, and the amount of oxidized hemoglobin increases. The signal processing circuit 30 detects, for example, an increase in the cerebral blood flow volume or an increase in the amount of oxidized hemoglobin and can thus estimate the degree of concentration or the emotional state of the subject.

A variety of combinations of wavelengths are possible. The light with a wavelength of 805 nm has an equal absorption amount for oxidized hemoglobin and for reduced hemoglobin. Therefore, when light at a wavelength of less than 805 nm and light at a wavelength of greater than 805 nm are combined, information on each of oxidized hemoglobin and reduced hemoglobin can be acquired. Furthermore, in addition to the stated two wavelengths, three wavelengths including the wavelength of 805 nm can be used. The use of three wavelengths necessitates three laser chips. However, this configuration makes it possible to obtain information from the third wavelength as well, and the use of this information can facilitate the computation.

The light source 1 may have a structure in which a plurality of light source packages are arranged. A single laser chip is embedded in each light source package. In this case, the optical element 3b and the diffuser 16 may be provided for each light source package.

The size of the forehead, serving as the portion 6 to be examined, in the X-direction is designated by W, and the size in the Y-direction is designated by h. Then, although there are individual differences, these sizes are, for example, approximately as follows: W=100 mm, and h=50 mm. As can be seen from FIGS. 14A and 14B, the curvature of the forehead in the X-direction is greater than the curvature of the forehead in the Y-direction. The distance from the surface of the optical element 3b to the portion 6 to be examined along the center axis 10 of the emission light is designated by WD. The distance from the surface of the optical element 3b to a peripheral portion in the X-direction of the portion 6 to be examined that is irradiated with the light 8 is designated by WD2. The distance from the surface of the optical element 3b to a peripheral portion in the Y-direction of the irradiated portion 6 to be examined is designated by WD1. When the portion 6 to be examined is a forehead, WD2>WD1 holds. The optical path length increases at the peripheral portion of the light 8 due to the curve of the forehead, and the optical intensity decreases. Therefore, the optical intensity tends to be lower at the peripheral portions of the portion 6 to be examined in the X-direction than in the Y-direction.

The present inventors have found the above-described characteristics of the optical intensity distribution held when the portion 6 to be examined is a forehead and examined a configuration of the optical element 3b suitable for these characteristics. As a result, the present inventors have conceived of the use of the optical element 3b that has a shape that can be approximated to an ellipse having a minor axis extending in the X-direction, in which the sag amount has a local minimum value at the center portion C, or the optical axis center, and a local maximum value at a portion other than the center portion C, and in which the shape of the locus 14a obtained by connecting the portions at which the sag amount is locally maximum can be approximated to an ellipse. Making the direction in which the curvature of the portion 6 to be examined is large (the X-direction in the drawings) coincide with the minor axis direction of the ellipse makes it possible to bring the illuminance distribution on the portion 6 to be examined closer to being more uniform.

It has been found that, when the portion 6 to be examined is a forehead, although there are individual differences, it suffices that a lens in which the eccentricity e of the above-described ellipse satisfies 0<e<0.6 be used.

In the present embodiment as well, the optical element 3b may include a lens array. The sag amount of each lens has a local minimum value at the center portion C, or the optical axis center, and a local maximum value at a portion other than the center portion C, or the optical axis center. The shape of the locus 14a obtained by connecting the local maximum portions can be approximated to an ellipse.

The shape of the locus 14a obtained by connecting the local maximum portions is not limited to an ellipse. The accurate shape of the locus 14a obtained when the sag amount of the optical element 3b can be expressed as a function of x and y including the term α₁ₓx²+α₂ₓx⁴+α_{1y}y²+α_{2y}y⁴ can be derived mathematically. The shape of the locus 14a can be approximated to an ellipse as described above and can also be a rhombus or a rhombus with four rounded corners, for example, depending on the conditions.

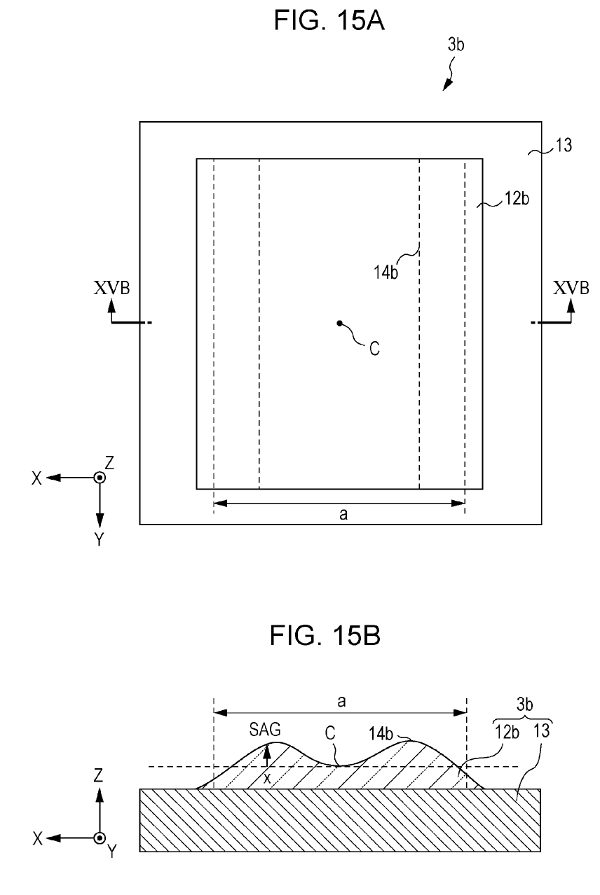

FIG. 15A is a plan view schematically illustrating a configuration of an optical element according to a modification of the third embodiment of the present disclosure. FIG. 15B is a sectional view taken along the XVB-XVB line indicated in FIG. 15A.

A difference from the optical elements according to the foregoing embodiments is that the thickness of a lens 12b in the optical element 3b is substantially constant in the Y-direction that is orthogonal to both the X-direction and the thickness direction of the lens 12b.

The optical element 3b according to the present modification increases the intensity of the light at the peripheral portions in the X-direction. The optical element 3b does not change the optical intensity distribution in the Y-direction.

As illustrated in FIG. 15B, the section of the lens 12b along a plane parallel to the XZ-plane has a concave lens shape at the center portion and therearound and a convex lens shape at the peripheral region. The optical element 3b disperses light, of the light emitted from the light source 1, incident on the center portion toward the peripheral portion and condenses light incident on the peripheral portion only in the X-direction. In other words, the optical element 3b functions to increase the intensity of the light 8 at the peripheral portion in the A-A plane where the portion 6 to be examined is present. Although not illustrated, the sections of the lens 12b along a plane parallel to the YZ-plane each have a shape of which the thickness is uniform. Thus, the shape of a locus 14b obtained by connecting the local maximum portions that lie on lines connecting the center portion C of the lens 12b and points on the outer periphery is a straight line.

The sag amount of the lens 12b in such an optical element 3b can be expressed as a function of x including α₁ₓx²+α₂ₓx⁴, in which the center portion C, or the optical axis center, serves as the origin, α₁ₓ is a positive real number, and α₂ₓ is a negative real number. The use of such an optical element 3b also makes it possible to constitute a biological information measuring apparatus suitable when the portion 6 to be examined is a forehead.

An optical element that increases the intensity of the transmitted light at peripheral portions not in the X-direction but in the Y-direction alone may instead be used. The sag amount of such an optical element can be expressed as a function of y including α_{1y}y²+α_{2y}y⁴, in which the optical axis center of the element serves as the origin, α_{1y} is a positive real number, and α_{2y} is a negative real number.

In the biological information measuring apparatus according to the present modification, the X-direction in which the optical element 3b increases the optical intensity at the peripheral portions is made to coincide with the lateral direction in which the curvature of the forehead is large, and thus an advantageous effect of reducing a decrease in the S/N of a biological signal from the peripheral region can be obtained.

In the first through third embodiments and the modifications thereof, an optical element in which primarily a lens has a nonuniform thickness is used. In place of the thickness of the lens or in addition to the thickness of the lens, an optical element in which the refractive index of a lens is nonuniform may be used, and still a similar advantageous effect can be obtained.

The present disclosure is not limited to the foregoing embodiments. A biological information measuring apparatus obtained by combining configurations of the biological information measuring apparatuses of the embodiments is also encompassed by the present disclosure, and a similar advantageous effect can be obtained.

What is claimed is:

1. A biological information measuring apparatus, comprising:
a light source that emits emission light with which a portion to be examined is to be irradiated;
a photodetector that detects light returning from the portion to be examined, the light being produced when the portion to be examined is irradiated with the emission light; and
an optical element that includes at least one lens, the optical element being disposed in an optical path between the light source and the portion to be examined, wherein
at least one value selected from the group consisting of a thickness of the at least one lens and a refractive index of the at least one lens varies along a first direction extending from a center portion toward an outer edge portion, the center portion being a portion that includes a center of the at least one lens, and
the at least one value is locally minimum at the center portion and locally maximum at a first portion that lies between the center portion and the outer edge portion.

2. The biological information measuring apparatus according to claim 1,
wherein the at least one value monotonically increases from the center portion toward the first portion and monotonically decreases from the first portion toward the outer edge portion.

3. The biological information measuring apparatus according to claim 1, wherein
the at least one lens includes a plurality of first portions, each of the plurality of first portions being the first portion,
one of the plurality of first portions is present between the center portion and each of a plurality of points on the outer edge portion, and
a locus obtained by connecting the plurality of first portions has a shape of one of a circle, an ellipse, and a rhombus.

4. The biological information measuring apparatus according to claim 1,
wherein the at least one value is constant in a second direction orthogonal to both the first direction and a thickness direction of the at least one lens.

5. The biological information measuring apparatus according to claim 1, wherein
the at least one lens includes a concave-convex surface that is concave at the center portion and convex at the first portion, and
the emission light incident on the at least one lens from the light source exits through the concave-convex surface.

6. The biological information measuring apparatus according to claim 1, further comprising:
a diffuser disposed in an optical path between the light source and the optical element.

7. The biological information measuring apparatus according to claim 1, wherein
the at least one lens has a shape that is rotationally symmetric about an axis passing through the center, and
a sag amount is expressed as a function of r including a term
α₁r²+α₂r⁴,
the sag amount indicating a difference between the thickness of the at least one lens at the center and the thickness of the at least one lens at a position that is spaced apart from the center by a distance r along a plane perpendicular to the axis,
where α₁ is a positive real number and α₂ is a negative real number.

8. The biological information measuring apparatus according to claim 1, wherein
a sag amount is expressed as a function of x and y including a term
α₁ₓx²+α_{1y}y²+α₂ₓx⁴+α_{2y}y⁴,
the sag amount indicating a difference between the thickness of the at least one lens at the center and the thickness of the at least one lens at a position that is spaced apart from the center by a distance x in the first direction and by a distance y in a second direction orthogonal to both the first direction and a thickness direction of the at least one lens,
where α₁ₓ and α_{1y} are positive real numbers and α₂ₓ and α_{2y} are negative real numbers.

9. The biological information measuring apparatus according to claim 1, wherein
a sag amount is expressed as a function of x including a term
α₁ₓx²⁺+α₂ₓx⁴ or
as a function of y including a term
α_{1y}y²+α_{2y}y⁴,
the sag amount indicating a difference between the thickness of the at least one lens at the center and the thickness of the at least one lens at a position that is spaced apart from the center by a distance x in the first direction and by a distance y in a second direction orthogonal to both the first direction and a thickness direction of the at least one lens,
where α₁ₓ and α_{1y} are positive real numbers and α₂ₓ and α_{2y} are negative real numbers.

10. The biological information measuring apparatus according to claim 1, wherein
the at least one lens includes a plurality of lenses arrayed two-dimensionally along a plane intersecting a center axis of the emission light,
at least one value selected from the group consisting of a thickness of each of the plurality of lenses and a refractive index of each of the plurality of lenses varies along a direction extending from a center portion of each of the plurality of lenses toward an outer edge portion, the center portion being a portion that includes a center of each of the plurality of lens, and
the at least one value selected from the group consisting of the thickness of each of the plurality of lenses and the refractive index of each of the plurality of lenses is locally minimum at the center portion and locally maximum at a portion that lies between the center portion and the outer edge portion.

11. The biological information measuring apparatus according to claim 1, further comprising:
a control circuit,
wherein the control circuit
controls the light source and the photodetector and
generates information pertaining to a blood flow in the portion to be examined based on a signal indicating a quantity of the light detected by the photodetector.

12. The biological information measuring apparatus according to claim 1,
wherein the emission light is incident on the at least one lens as divergent light.

13. An optical element, comprising:
at least one lens, wherein
at least one value selected from the group consisting of a thickness of the at least one lens and a refractive index of the at least one lens varies along a first direction extending from a center portion toward an outer edge portion, the center portion being a portion that includes a center of the at least one lens, and
the at least one value is locally minimum at the center portion and locally maximum at a first portion that lies between the center portion and the outer edge portion.

14. The optical element according to claim 13,
wherein the at least one value monotonically increases from the center portion toward the first portion and monotonically decreases from the first portion toward the outer edge portion.

15. The optical element according to claim 13, wherein
the at least one lens includes a plurality of first portions, each of the plurality of first portions being the first portion,
one of the plurality of first portions is present between the center portion and each of a plurality of points on the outer edge portion, and
a locus obtained by connecting the plurality of first portions has a shape of one of a circle, an ellipse, and a rhombus.

16. The optical element according to claim 13,
wherein the at least one value is constant in a second direction orthogonal to both the first direction and a thickness direction of the at least one lens.

17. The optical element according to claim 13, wherein
the at least one lens includes a concave-convex surface that is concave at the center portion and convex at the first portion, and
the concave-convex surface is disposed at a position where light exits from the at least one lens.

18. The optical element according to claim 13, wherein
the at least one lens has a shape that is rotationally symmetric about an axis passing through the center, and
a sag amount is expressed as a function of r including a term
α₁r²+α₂r⁴,
the sag amount indicating a difference between the thickness of the at least one lens at the center and the thickness of the at least one lens at a position that is spaced apart from the center by a distance r along a plane perpendicular to the axis,
where α₁ is a positive real number and α₂ is a negative real number.

19. The optical element according to claim 13, wherein
a sag amount is expressed as a function of x and y including a term
α₁ₓx²+α_{1y}y²+α₂ₓx⁴+α_{2y}y⁴,
the sag amount indicating a difference between the thickness of the at least one lens at the center and the thickness of the at least one lens at a position that is spaced apart from the center by a distance x in the first direction and by a distance y in a second direction orthogonal to both the first direction and a thickness direction of the at least one lens,
where α₁ₓ and α_{1y} are positive real numbers and α₂ₓ and α_{2y} are negative real numbers.

20. The optical element according to claim 13, wherein a sag amount is expressed as a function of x including a term
α₁ₓx²+α₂ₓx⁴ or
as a function of y including a term
α_{1y}y²+α_{2y}y⁴,
the sag amount indicating a difference between the thickness of the at least one lens at the center and the thickness of the at least one lens at a position that is spaced apart from the center by a distance x in the first direction and by a distance y in a second direction orthogonal to both the first direction and a thickness direction of the at least one lens,
where α₁ₓ and α_{1y} are positive real numbers and α₂ₓ and α_{2y} are negative real numbers.

21. The optical element according to claim 13, wherein
the at least one lens includes a plurality of lenses arrayed two-dimensionally along a plane intersecting a center axis of the emission light,
at least one value selected from the group consisting of a thickness of each of the plurality of lenses and a refractive index of each of the plurality of lenses varies along a direction extending from a center portion of each of the plurality of lenses toward an outer edge portion, the center portion being a portion that includes a center of each of the plurality of lens, and
the at least one value selected from the group consisting of the thickness of each of the plurality of lenses and the refractive index of each of the plurality of lenses is locally minimum at the center portion and locally maximum at a portion that lies between the center portion and the outer edge portion.

22. An illumination apparatus, comprising:
a light source that emits emission light with which an object is to be irradiated; and
an optical element, wherein
the optical element includes at least one lens,
at least one value selected from the group consisting of a thickness of the at least one lens and a refractive index of the at least one lens varies along a first direction extending from a center portion toward an outer edge portion, the center portion being a portion that includes a center of the at least one lens,
the at least one value is locally minimum at the center portion and locally maximum at a first portion that lies between the center portion and the outer edge portion,
the at least one lens includes a concave-convex surface that is concave at the center portion and convex at the first portion, and
the emission light incident on the at least one lens from the light source exits through the concave-convex surface.

23. The illumination apparatus according to claim 22, further comprising:
a diffuser disposed in an optical path between the light source and the optical element.
